(19) United States
(12) Patent Application Publication
(10) Pub. No.: US 2021/0060167 A1
(43) Pub. Date: Mar. 4, 2021

(54) CARBON MONOXIDE-RELEASING MOLECULE CONJUGATES AND RELATED COMPOSITIONS AND METHODS

(71) Applicant: The Regents of the University of California, Oakland, CA (US)

(72) Inventors: Pradip Mascharak, Santa Cruz, CA (US); Brian Kawahara, Santa Cruz, CA (US)

(21) Appl. No.: 17/004,522

(22) Filed: Aug. 27, 2020

Related U.S. Application Data
(60) Provisional application No. 62/892,861, filed on Aug. 28, 2019.

(51) Int. Cl. A61K 47/54; A61K 47/68; A61K 47/64; A61P 35/00

(52) U.S. Cl. CPC A61K 47/545; A61P 35/00; A61K 47/64; A61K 47/6869

(57) ABSTRACT

Provided are conjugates comprising a carbon monoxide (CO)-releasing molecule (CORM) conjugated to a targeting moiety that binds to a cell surface molecule of a target cell. In certain embodiments, the CORM is a photoactivatable CORM (photoCORM). According to some embodiments, the target cell is a cancer cell, and the targeting moiety (e.g., an antibody) specifically binds a tumor antigen present on the cancer cell. Also provided are compositions comprising the conjugates of the present disclosure, and methods of using the conjugates of the present disclosure, e.g., to treat an individual having cancer.

### CROSS-REFERENCE TO RELATED APPLICATIONS

This application claims the benefit of U.S. Provisional Patent Application No. 62/892,861, filed Aug. 28, 2019, which application is incorporated herein by reference in its entirety.

### INTRODUCTION

Carbon monoxide (CO) is a cytoprotective and homeostatic molecule with important signaling capabilities in physiological and pathophysiological situations. The endogenous production of CO occurs through the activity of constitutive (haem oxygenase 2) and inducible (haem oxygenase 1) haem oxygenases, enzymes that are responsible for the catabolism of haem. Through the generation of its products, which in addition to CO includes the bile pigments biliverdin, bilirubin and ferrous iron, the haem oxygenase 1 system also has an obligatory role in the regulation of the stress response and in cell adaptation to injury.

CO has an important biological activity as a signaling molecule with marked protective actions against apoptosis and endothelial oxidative damage. Abnormal metabolism and function of CO contribute to the pathogenesis and development of cardiovascular diseases. Results have been reported in which CO and CO-releasing molecules (CORMs) prevent intimal hyperplasia by arresting hyperproliferative vascular smooth muscle cells and increased mobilization and recruitment of bone-marrow-derived progenitor cells. Clinical studies have demonstrated beneficial properties of CORMs in transplantation. The anti-inflammatory properties of CO and CORMs have been demonstrated in a multitude of animal models of inflammation, suggesting a possible therapeutic application for inflammatory diseases.

### SUMMARY

Provided are conjugates comprising a carbon monoxide (CO)-releasing molecule (CORM) conjugated to a targeting moiety that binds to a cell surface molecule of a target cell. In certain embodiments, the CORM is a photoactivatable CORM (photoCORM). According to some embodiments, the target cell is a cancer cell, and the targeting moiety (e.g., an antibody) specifically binds a tumor antigen present on the cancer cell. Also provided are compositions comprising the conjugates of the present disclosure, and methods of using the conjugates of the present disclosure, e.g., to treat an individual having cancer.

### BRIEF DESCRIPTION OF THE FIGURES

FIG. 1 Structure of a photoCORM according to one embodiment of the present disclosure (Complex 1).

FIG. 2 Complex 2: streptavidin-conjugated IgG. Panel A: Native protein gel electrophoresis of crude Complex 2. Panel B: Size-exclusion chromatograms of fractions of Complex 2. [Retention time, molecular weight, identity] (i) [35.4 min, ˜210 kDa, IgG+1 streptavidin]. (ii) [32.2 min, ˜260 kDa, IgG+2 streptavidin]. (iii) [29.9 min, ˜313 kDa, IgG+3 streptavidin]. (iv) [28.6 min, ˜366 kDa, IgG+4 streptavidin].

FIG. 3 Synthesis and characterization of the antibody-photoCORM conjugate (Ab-photoCORM) and proteomic analysis of the Ab-photoCORM. The scheme of bottom-up proteomics of the Ab-photoCORM is also shown.

FIG. 4 Proteomic scores of the Ab-photoCORMs synthesized in this study. Biotin-photoCORM (Complex 1) was observed in the full MS scan of the tryptic digest of Ab-photoCORM. Protein scores greater than 67 are significant (i.e. p<0.05).

FIG. 5 Antibody-photoCORM conjugates (Ab-photoCORMs) deliver cytotoxic levels to ovarian cancer cell lines via immunosorbent assay. Panel A: Western analysis of whole cell lysates of cell lines OVCAR-5 and SKOV-3, probing for antigens recognized by a family of Ab-photoCORMs. Panel B: Cell viability, as measured by cellular reduction of MTT, of OVCAR-5 and SKOV-3 24 h post-immunosorbent assay utilizing 2 μg/mL Ab-photoCORM conjugates. Panel C: Dose-dependency of α-HCAM-photoCORM, compared to α-Control-photoCORM, on cell viability. Data representative of n=3 independent experiments. (* p<0.05)

### DETAILED DESCRIPTION

Before the conjugates, compositions and methods of the present disclosure are described in greater detail, it is to be understood that the conjugates, compositions and methods are not limited to particular embodiments described, as such may, of course, vary. It is also to be understood that the terminology used herein is for the purpose of describing particular embodiments only, and is not intended to be limiting, since the scope of the conjugates, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the conjugates, compositions and methods. The upper and lower limits of these smaller ranges may independently be included in the smaller ranges and are also encompassed within the conjugates, compositions and methods, subject to any specifically excluded limit in the stated range. Where the stated range includes one or both of the limits, ranges excluding either or both of those included limits are also included in the conjugates, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the conjugates, compositions and methods belong. Although any conjugates, compositions and methods similar or equivalent to those described herein can also be used in the practice or testing of the conjugates, compositions and methods, representative illustrative conjugates, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present conjugates, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the conjugates, compositions and methods, which are, for clarity, described in the context of separate embodiments, may also be provided in combination in a single embodiment. Conversely, various features of the conjugates, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present conjugates, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Conjugates

The present disclosure provides conjugates. According to some embodiments, the conjugates comprise a targeting moiety that binds to a cell surface molecule of a target cell, and a carbon monoxide (CO)-releasing molecule (CORM) conjugated to the targeting moiety. As demonstrated in the Experimental section herein, antigen-enhanced delivery of carbon monoxide (CO) was achieved by linking CO-releasing molecules to a variety of antigen-recognizing targeting moieties. The synthesized targeting moiety-CORM conjugates were capable of delivering cytotoxic levels of CO to cancer cells. Details regarding the conjugates of the present disclosure will now be described.

Targeting Moieties

As summarized above, a conjugate of the present disclosure includes a targeting moiety that binds to a cell surface molecule of a target cell. The targeting moiety may vary and may be selected based, e.g., on the nature of the cell surface molecule on the target cell. Non-limiting examples of a targeting moiety that may be employed include a polypeptide, an antibody, a ligand, an aptamer, a nanoparticle, and a small molecule.

In certain embodiments, the targeting moiety specifically binds the cell surface molecule of the target cell. As used herein, a first molecule “specifically binds” to a second molecule if it binds to or associates with the second molecule with an affinity or Kₐ (that is, an equilibrium association constant of a particular binding interaction with units of 1/M) of, for example, greater than or equal to about 10⁵ M⁻¹. In certain embodiments, the first molecule binds to the second molecule with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the first molecule for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

According to some embodiments, the targeting moiety is an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, Fv, single chain antibody, scFv, Fab, F(ab′)₂, or Fab′. The antibody may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

An antibody included in the conjugate will vary based on the cell to be targeted. In some embodiments, the antibody specifically binds to an antigen on the surface of a target cell. Target cells of interest include, but are not limited to, cells that are relevant to a particular disease or condition. According to some embodiments, the target cell is selected from a cancer cell, an immune cell, and an endothelial cell. As such, in some embodiments, the target cells are cancer cells. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In certain aspects, the cancer cell is a carcinoma cell.

In certain embodiments, when the target cell is a cancer cell, the antibody of the conjugate specifically binds to a tumor antigen on the surface of the cancer cell. Non-limiting examples of tumor antigens to which the antibody of the ADC may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), EpCAM, ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GLUT3, glycoprotein non-metastatic B (GPNMB), guanylate cyclase 2 C (GUCY2C), HCAM, human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), trophoblast cell-surface antigen (TROP-2), and VEGF-A.

Non-limiting examples of antibodies that specifically bind to tumor antigens which may be employed in a conjugate of the present disclosure include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sotituzumab, Catumaxomab, Ertumaxomab, Depatuxizumab, Ontuxizumab, Blontuvetmab, Tamtuvetmab, or a tumor antigen-binding variant thereof. As used herein, “variant” is meant the antibody specifically binds to the particular antigen (e.g., HER2 for trastuzumab) but has fewer or more amino acids than the parental antibody (e.g., is a fragment (e.g., scFv) of the parental antibody), has one or more amino acid substitutions relative to the parental antibody, or a combination thereof.

In some embodiments, an antibody of the conjugate is an antibody approved by the United States Food and Drug Administration and/or the European Medicines Agency (EMA) for use as a therapeutic antibody (e.g., for targeting certain disease-associated cells in a patient, etc.), or a fragment thereof (e.g., a single-chain version of such an antibody, such as an scFv version of the antibody) that retains the ability to specifically bind the target antigen.

When a conjugate of the present disclosure employs an antibody as the targeting moiety, the CORM may be conjugated to any convenient portion of the antibody. In certain embodiments, the CORM is conjugated to a light chain of the antibody, e.g., a kappa (κ) light chain or fragment thereof or a lambda (λ) light chain or fragment thereof. According to some embodiments, the antibody light chain or fragment thereof includes a light chain variable region (V_{L}). Such an antibody light chain or fragment thereof may further include an antibody light chain constant region (C_{L}) or fragment thereof. In certain embodiments, the antibody light chain or fragment thereof is a full-length antibody light chain—that is, an antibody light chain that includes a V_{L} and a C_{L}. In certain embodiments, the CORM is conjugated to a V_{L} (if present) or a C_{L} (if present), e.g., at or near the N-terminus of a V_{L} or at or near the C-terminus of a C_{L}.

When a conjugate of the present disclosure employs an antibody as the targeting moiety, the CORM may be conjugated to a heavy chain or fragment thereof of the antibody. In certain embodiments, the antibody heavy chain or fragment thereof includes a γ, α, δ, ϵ, or μ antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain variable region (V_{H}). Such an antibody heavy chain or fragment thereof may further include a heavy chain constant region or fragment thereof. For example, when the antibody includes a heavy chain constant region or fragment thereof, the antibody heavy chain constant region or fragment thereof may include one or more of a C_{H}1 domain, C_{H}2 domain, and/or C_{H}3 domain. According to some embodiments, the antibody heavy chain is a full-length antibody heavy chain—that is, an antibody heavy chain that includes a V_{H}, a C_{H}1 domain, a C_{H}2 domain, and a C_{H}3 domain. In certain embodiments, the CORM is conjugated to an Fc region of the antibody. According to some embodiments, the CORM is conjugated to the antibody at or near the N-terminus of a V_{H} or at or near the C-terminus of a C_{H}3 domain.

According to certain embodiments, the targeting moiety is a ligand. As used herein, a “ligand” is a substance that forms a complex with a biomolecule to serve a biological purpose. The ligand may be a substance selected from a circulating factor, a secreted factor, a cytokine, a growth factor, a hormone, a peptide, a polypeptide, a small molecule, and a nucleic acid, that forms a complex with the cell surface molecule on the surface of the target cell. In certain aspects, when the targeting moiety is a ligand, the ligand is modified in such a way that complex formation with the cell surface molecule occurs, but the normal biological result of such complex formation does not occur. In certain aspects, the ligand is the ligand of a cell surface receptor present on the target cell. Cell surface receptors of interest include, but are not limited to, receptor tyrosine kinases (RTKs), non-receptor tyrosine kinases (non-RTKs), growth factor receptors, etc. When the conjugates of the present disclosure include a ligand as the targeting moiety, the CORM may be conjugated to any suitable region of the ligand, e.g., a region of attachment that does not interfere or substantially interfere with the ability of the ligand to bind (e.g., specifically bind) the target cell surface molecule.

In certain embodiments, the targeting moiety is an aptamer. By “aptamer” is meant a nucleic acid (e.g., an oligonucleotide) that has a specific binding affinity for the target cell surface molecule. Aptamers exhibit certain desirable properties for targeted delivery of the CORM, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C). Aptamers that find use in the conjugates of the present disclosure include those described in Zhu et al. (2015) ChemMedChem 10(1):39-45; Sun et al. (2014) Mol. Ther. Nucleic Acids 3:e182; and Zhang et al. (2011) Curr. Med. Chem. 18(27):4185-4194.

According to some embodiments, the targeting moiety is a nanoparticle. As used herein, a “nanoparticle” is a particle having at least one dimension in the range of from 1 nm to 1000 nm, from 20 nm to 750 nm, from 50 nm to 500 nm, including 100 nm to 300 nm, e.g., 120-200 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain aspects, the nanoparticle includes on its surface one or more of the other targeting moieties described herein, e.g., antibodies, ligands, aptamers, small molecules, etc. Nanoparticles that find use in the conjugates of the present disclosure include those described in Wang et al. (2010) Pharmacol. Res. 62(2):90-99; Rao et al. (2015) ACS Nano 9(6):5725-5740; and Byrne et al. (2008) Adv. Drug Deliv. Rev. 60(15):1615-1626.

In certain aspects, the targeting moiety is a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In certain aspects, the target cell surface molecule is a receptor for which the ligand is a small molecule, and the small molecule of the conjugate is the small molecule ligand (or a derivative thereof) of the receptor. Small molecules that find use in targeting a conjugate to a target cell of interest are known. As just one example, folic acid (FA) derivatives have been shown to effectively target certain types of cancer cells by binding to the folate receptor, which is overexpressed, e.g., in many epithelial tumors. See, e.g., Vergote et al. (2015) Ther. Adv. Med. Oncol. 7(4):206-218. In another example, the small molecule sigma-2 has proven to be effective in targeting cancer cells. See, e.g., Hashim et al. (2014) Molecular Oncology 8(5):956-967. Sigma-2 is the small molecule ligand for sigma-2 receptors, which are overexpressed in many proliferating tumor cells including pancreatic cancer cells. In certain aspects, a conjugate of the present disclosure includes a small molecule as the targeting moiety, in which it has been demonstrated in the context of a small molecule drug conjugate (SMDC) that the small molecule is effective at targeting a conjugate to a target cell of interest by binding to a cell surface molecule on the target cell.

Carbon Monoxide (CO)-Releasing Molecules (CORMs)

As summarized above, a conjugate of the present disclosure includes a carbon monoxide (CO)-releasing molecule (CORM) conjugated to the targeting moiety. By “carbon monoxide-releasing molecule” or “CORM” is meant a chemically-bound form of CO as a prodrug for physiological CO release.

A large variety of CORMs may be employed in the conjugates of the present disclosure. Due to the strong but modifiable binding of CO to transition metal centers, metal carbonyl complex-based CORMs have been developed. By proper selection of the metal center, the number and spatial arrangement of CO ligands around it, and the nature of any co-ligands completing the preferred coordination sphere of the metal, it is possible to tune the CO-release properties in a wide range. See, e.g., Schatzschneider, U. (2015) Br J Pharmacol. 172(6):1638-50. The “inner” part of such a molecule has been termed the CORM sphere or coordination sphere by Romao et al. (2012) Chem Soc Rev 41: 3571-3583. Key parameters are the number of CO molecules that can be released from the metal coordination sphere, the kinetics of the CO release process and the trigger mechanism required to initiate the liberation of the CO. Other types of CORMs that may be employed include main group boranocarbonate-based CORMs, purely organic CORMS (e.g., based on unsaturated cyclic 1,2-diketones, xanthene-9-carboxylic acid, or the like), etc.

In certain embodiments, the CORM is [RuCl(glycinato)(CO)₃]. According to some embodiments, the CORM is [RuCl(μ-Cl)(CO)₃]₂. In certain embodiments, the CORM comprises a molybdenum(0) tricarbonyl complexes of the general formula [Mo(CO)₃(L)₃], in which L is a neutral monodentate ligand.

According to some embodiments, the CORM is a prodrug that is stable in serum and triggered (e.g., in the targeted tissue) by a specific stimulus. For example, the CORM may be an enzyme-triggered CORM (ET-CORM). See, e.g., Romanski et al. (2011) Angew Chem Int Ed 50:2392-2396; Romanski et al. (2012) Dalton Trans 41:13862-13875; Romanski et al. (2012) Organometallics 31:5800-5809; etc.

According to some embodiments, the CORM is a photoactivatable CORM (photoCORM), where by “photoactivatable” is meant the external trigger is based on the use of light to induce CO release from a transition metal carrier system. In certain embodiments, a conjugate of the present disclosure comprises the photoCORM [Mn(CO)₃(phen)(4-pyAl)](CF₃SO₃), wherein phen is 1,10-phenanthroline, and 4-pyAl is pyridine-4-carboxaldehyde. According to some embodiments, a conjugate of the present disclosure comprises a photoCORM selected from [Mn(CO)₃(pbt)(PTA)]CF₃SO₃ (where PTA=1,3,5-triaza-7-phosphaadamantane); [Mn(CO)₃(phen)(PTA)]CF₃SO₃; [Re(CO)₃(pbt)(PTA)]CF₃SO₃; [Re(CO)₃(phen)(PTA)]CF₃SO₃; [Re(CO)₃(pbt)(PPh₃)](CF₃SO₃); [Re(CO)₃(phen)(pyAl)](CF₃SO₃); [Mn(Imdansyl)(CO)₃(phen)](CF₃SO₃); [Re(Imdansyl)(CO)₃(phen)](CF₃SO₃); [Re(bpy)(CO)₃(PR₃)]⁺, (where R═CH2OH); and fac-[MnBr(CO)₃(pbt)]. Details regarding photoCORMs may be found, e.g., in Wright et al. (2016) Dalton Trans 45(16):6801-11; Gonzales et al. (2014) J lnorg Biochem. 133:127-35; and elsewhere.

Conjugation, Linkers and Methods of Making Conjugates

A wide variety of approaches may be employed to conjugate the CORM to the targeting moiety, and such approaches may vary depending upon the particular CORM and targeting moiety selected for the conjugate.

In some embodiments, the CORM is covalently conjugated to the targeting moiety. When the CORM is covalently conjugated to the targeting moiety, the CORM may be conjugated to the targeting moiety via a linker. Linkers that find use in the conjugates of the present disclosure include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof.

In certain aspects, the CORM is covalently conjugated to the targeting moiety via a linker, and the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13.

According to some embodiments, the CORM is conjugated to the targeting moiety via a non-cleavable linker. A non-limiting example of a non-cleavable linker is a thioether linker. In certain embodiments, the thioether linker is a succinimidyl 4-(N-maleimidomethyl)cyclohexane-1-carboxylate (SMCC) linker.

Numerous strategies are available for linking the CORM to the targeting moiety via a linker. For example, a CORM may be derivatized by covalently attaching the linker to the CORM, where the linker has a functional group capable of reacting with a “chemical handle” on the targeting moiety. The functional group on the linker may vary and may be selected based on compatibility with the chemical handle on the targeting moiety. According to one embodiment, the chemical handle on the targeting moiety is provided by incorporation of an unnatural amino acid having the chemical handle into the targeting moiety. Unnatural amino acids may be incorporated via chemical synthesis or recombinant approaches (e.g., using a suitable orthogonal amino acyl tRNA synthetase-tRNA pair for incorporation of the unnatural amino acid during translation in a host cell).

In other aspects, the chemical handle on the targeting moiety is provided using an approach that does not involve an unnatural amino acid. A targeting moiety containing no unnatural amino acids could be conjugated to a CORM or CORM-linker construct by utilizing, e.g., nucleophilic functional groups of the targeting moiety (such as the N-terminal amine or the primary amine of lysine, or any other nucleophilic amino acid residue) as a nucleophile in a substitution reaction with a CORM-linker construct bearing a reactive leaving group or other electrophilic group. An example would be to prepare a CORM-linker construct bearing an N-hydroxysuccinimidyl (NHS) ester and allow it to react with the targeting moiety under aqueous conditions at elevated pH (˜10) or in polar organic solvents such as DMSO with an added non-nucleophilic base, such as N,N-diisopropylethylamine.

The functional group of an unnatural amino acid present in the targeting moiety may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the targeting moiety and the linker portion of a linker-CORM moiety may include an alkyne functional group, such that the targeting moiety and linker-CORM moiety are covalently conjugated via azide-alkyne cycloaddition.

In certain embodiments, the CORM is non-covalently conjugated to the targeting moiety. In one non-limiting example of non-covalent conjugation, the CORM is conjugated to the targeting moiety via a streptavidin-biotin interaction. By way of example, the streptavidin-biotin interaction may be provided by employing a CORM that is biotinylated and a targeting moiety conjugated to streptavidin. Examples of non-covalent conjugation via streptavidin-biotin interaction which may be employed in the conjugates of the present disclosure are provided in the Experimental section below.

Accordingly, aspects of the present disclosure include methods of making the conjugates of the present disclosure. In certain embodiments, the methods include conjugating a carbon monoxide (CO)-releasing molecule (CORM) to a targeting moiety (e.g., an antibody) that binds to a cell surface molecule on the surface of a target cell, e.g., a tumor antigen present on the surface of a cancer cell. For example, such methods may include covalently conjugating the CORM to the targeting moiety. In certain embodiments, the methods include covalently conjugating the CORM to the targeting moiety via a linker. According to some embodiments, the methods include non-covalently conjugating the CORM to the targeting moiety. In certain embodiments, the conjugating comprises site-specifically conjugating the CORM to a non-natural amino acid of the targeting moiety. According to some embodiments, the methods include conjugating a photoCORM to the targeting moiety, e.g., an antibody.

It will be appreciated that the particular approach for attaching, e.g., a linker to the CORM and the linker to the targeting moiety (or vice versa) will vary depending upon the particular CORM and functional groups selected and employed in the linker and targeting moiety.

### Compositions

Also provided are compositions that include a conjugate of the present disclosure. The compositions may include any of the conjugates described herein, e.g., a conjugate having any of the targeting moieties and CORMs described herein. In certain aspects, the compositions include a conjugate of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a ribonuclease inhibitor, glycerol, a chelating agent, and the like may be present in such compositions.

Pharmaceutical compositions are also provided. The pharmaceutical compositions include any of the conjugates of the present disclosure, and a pharmaceutically acceptable carrier. The pharmaceutical compositions generally include a therapeutically effective amount of the conjugate. By “therapeutically effective amount” is meant a dosage sufficient to produce a desired result, e.g., an amount sufficient to effect beneficial or desired therapeutic (including preventative) results, such as a reduction in a symptom of a disease or disorder associated with the target cell or a population thereof, as compared to a control. An effective amount can be administered in one or more administrations.

A conjugate of the present disclosure can be incorporated into a variety of formulations for therapeutic administration. More particularly, the conjugate can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the conjugates of the present disclosure suitable for administration to a patient (e.g., suitable for human administration) are generally sterile and may further be free of detectable pyrogens or other contaminants contraindicated for administration to a patient according to a selected route of administration.

In pharmaceutical dosage forms, the conjugates can be administered in the form of their pharmaceutically acceptable salts, or they may also be used alone or in appropriate association, as well as in combination, with other pharmaceutically active compounds, e.g., an anti-cancer agent (including but not limited to small molecule anti-cancer agents), an immune checkpoint inhibitor, and any combination thereof. The following methods and carriers/excipients are merely examples and are in no way limiting.

For oral preparations, the conjugate can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The conjugate can be formulated for parenteral (e.g., intravenous, intra-arterial, intraosseous, intramuscular, intracerebral, intracerebroventricular, intrathecal, subcutaneous, etc.) administration. In certain aspects, the conjugate is formulated for injection by dissolving, suspending or emulsifying the conjugate in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the conjugate may be prepared by mixing the conjugate having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the conjugate may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof.

In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the conjugate against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes a conjugate of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods of Use

Aspects of the present disclosure also include methods of using the conjugates of the present disclosure. The methods find use in a variety of applications, including research applications and clinical applications.

In certain embodiments, provided are methods comprising administering to an individual in need thereof an effective amount of a pharmaceutical composition of the present disclosure. The pharmaceutical compositions employed in the methods may include any of the conjugates of the present disclosure, including any of the conjugates described in the Conjugates section above, which conjugates are incorporated but not reiterated herein for the sake of brevity.

According to some embodiments, provided are methods of treating cancer. Such methods comprise administering to an individual having cancer an effective amount of a pharmaceutical composition of the present disclosure, wherein the CORM (e.g., photoCORM) is conjugated to a targeting moiety that specifically binds a tumor antigen present on cells of the individual's cancer. In certain embodiments, the individual has a cancer characterized by the presence of a solid tumor, a semi-solid tumor, a primary tumor, a metastatic tumor, a liquid tumor (e.g., a leukemia or lymphoma), and/or the like. According to some embodiments, the individual has a cancer selected from breast cancer, glioblastoma, neuroblastoma, head and neck cancer, gastric cancer, ovarian cancer, skin cancer (e.g., basal cell carcinoma, melanoma, or the like), lung cancer, colorectal cancer, prostate cancer, glioma, bladder cancer, endometrial cancer, kidney cancer, leukemia (e.g., T-cell acute lymphoblastic leukemia (T-ALL), acute myeloid leukemia (AML), etc.), liver cancer (e.g., hepatocellular carcinoma (HCC), such as primary or recurrent HCC), a B-cell malignancy (e.g., non-Hodgkin lymphomas (NHL), chronic lymphocytic leukemia (CLL), follicular lymphoma, mantle cell lymphoma, diffuse large B-cell lymphoma, and the like), pancreatic cancer, thyroid cancer, any combinations thereof, and any sub-types thereof.

By “treating” or “treatment” is meant at least an amelioration of the symptoms associated with the pathological condition of the individual (e.g., cancer), where amelioration is used in a broad sense to refer to at least a reduction in the magnitude of a parameter, e.g. symptom, associated with the condition (e.g., cancer) being treated. As such, treatment also includes situations where the condition, or at least symptoms associated therewith, are completely inhibited, e.g., prevented from happening, or stopped, e.g., terminated, such that the individual no longer suffers from the condition, or at least the symptoms that characterize the condition.

The pharmaceutical composition is administered to the individual in a therapeutically effective amount. In some embodiments, a therapeutically effective amount of the conjugate is an amount that, when administered alone (e.g., in monotherapy) or in combination (e.g., in combination therapy) with one or more additional therapeutic agents, in one or more doses, is effective to reduce the symptoms of the pathological condition (e.g., cancer) in the individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the conjugate. According to some embodiments, when the individual has cancer, the methods of the present disclosure inhibit growth, metastasis and/or invasiveness of cancer cells of the individual's cancer when the conjugate is administered in an effective amount.

Dosing is dependent on severity and responsiveness of the condition (e.g., cancer) to be treated. Optimal dosing schedules can be calculated from measurements of drug accumulation in the body of the individual. The administering physician can determine optimum dosages, dosing methodologies and repetition rates. Optimum dosages may vary depending on the relative potency of individual agent and can generally be estimated based on EC₅₀s found to be effective in in vitro and in vivo animal models, etc. In general, dosage is from 0.01 pg to 100 g per kg of body weight, and may be given once or more daily, weekly, monthly or yearly. The treating physician can estimate repetition rates for dosing based on measured residence times and concentrations of the conjugate in bodily fluids or tissues. Following successful treatment, it may be desirable to have the individual undergo maintenance therapy to prevent the recurrence of the disease state, where the conjugate is administered in maintenance doses, ranging from 0.01 μg to 100 g per kg of body weight, once or more daily, to once every several months, once every six months, once every year, or at any other suitable frequency.

The agent may be administered to an individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, oral, and other enteral and parenteral routes of administration. Routes of administration may be combined, if desired, or adjusted depending upon the particular agent and/or the desired effect. The agent may be administered in a single dose or in multiple doses. In some embodiments, the agent is administered parenterally, e.g., intravenously, intra-arterially, or the like. In some embodiments, the agent is administered by injection, e.g., for systemic delivery (e.g., intravenous infusion) or to a local site, e.g., intratumoral injection.

In certain embodiments, the conjugate administered to the individual comprises an in vivo activatable CORM, and the methods further include, subsequent to administering the activatable CORM to the individual, activating the CORM in vivo. The type of activation will vary depending upon the type of activation to which the CORM is designed to respond. Activation may be enzymatic activation (when the CORM is an enzyme-activatable CORM), photoactivation (when the CORM is a photoCORM), etc.

According to some embodiments, the conjugate comprises a photoactivatable CORM, and subsequent to administering the pharmaceutical composition to the individual and allowing the conjugate to reach the target tissue (i.e., tumor tissue comprising tumor cells that express a cell surface tumor antigen to which the targeting moiety specifically binds), the methods further include irradiating the CORM with electromagnetic radiation suitable to activate the CORM, thereby causing CO release from the CORM in vivo at the target tissue to destroy cells of the tissue. The type of electromagnetic radiation will vary depending upon the type of electromagnetic radiation suitable to activate the particular photoCORM present in the conjugate. Details regarding in vivo activatable CORMs for delivery of therapeutic CO in vivo are found, e.g., in Garcia-Gallego et al. (2014) Angew Chem Int Ed Engl. 53(37):9712-21, the disclosure of which is incorporated herein by reference in its entirety for all purposes.

The individual to whom the pharmaceutical composition is administered may vary. In certain embodiments, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). According to some embodiments, the individual is a human. In certain embodiments, the individual is an animal model (e.g., a mouse model, a primate model, or the like) of a human pathological condition, e.g., cancer.

### Kits

As summarized above, the present disclosure also provides kits. In certain embodiments, the kits find use in practicing the methods of the present disclosure. According to some embodiments, a kit of the present disclosure includes any of the conjugates of the present disclosure, and instructions for contacting cells with the conjugate to induce death of the cells. The contacting may be in vitro or in vivo. Such kits may include any of the conjugates of the present disclosure, including any of the conjugates described in the Conjugates section above, which description is incorporated but not reiterated herein for purposes of brevity. When the conjugate includes an activatable CORM, the instructions may further include instructions for activating the CORM.

In certain embodiments, provided are kits that include a pharmaceutical composition of the present disclosure, and instructions for administering an effective amount of the pharmaceutical composition to an individual in need thereof. Such kits may include a quantity of the conjugate, present in unit dosages, e.g., ampoules, or a multi-dosage format. As such, in certain embodiments, the kits may include one or more (e.g., two or more) unit dosages (e.g., ampoules) of a composition that includes the agent. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular conjugate employed, the effect to be achieved, and the pharmacodynamics associated with the conjugate, in the individual. Accordingly, in certain embodiments, the kit includes the pharmaceutical composition present in one or more unit dosages, e.g., two or more unit dosages. In yet other embodiments, the kits may include a single multi dosage amount of the composition.

According to some embodiments, the individual has cancer, the targeting moiety specifically binds a tumor antigen present on cells of the cancer, and the kit includes instructions for administering to the individual a therapeutically effective amount of the pharmaceutical composition to treat the cancer. When the CORM is an activatable (e.g., photoactivatable) CORM, the instructions may further include instructions for activating the CORM (e.g., photoactivating using a device comprising a suitable source of electromagnetic radiation) in vivo to induce death of the cancer cells present in the individual.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:

1. A conjugate, comprising:
a targeting moiety that binds to a cell surface molecule of a target cell; and
a carbon monoxide (CO)-releasing molecule (CORM) conjugated to the targeting moiety.

2. The conjugate of embodiment 1, wherein the targeting moiety is selected from the group consisting of: a polypeptide, an antibody, a ligand, an aptamer, a nanoparticle, and a small molecule.

3. The conjugate of embodiment 2, wherein the targeting moiety is an antibody.

4. The conjugate of embodiment 3, wherein the antibody is a monoclonal antibody.

5. The conjugate of embodiment 3 or embodiment 4, wherein the antibody is a humanized or human antibody.

6. The conjugate of any one of embodiments 3 to 5, wherein the antibody is selected from the group consisting of: an IgG, Fv, single chain antibody, scFv, Fab, F(ab')2, or Fab'.

7. The conjugate of embodiment 6, wherein the antibody is an IgG1.

8. The conjugate of any one of embodiments 3 to 7, wherein the CORM is conjugated to a light chain of the antibody.

9. The conjugate of any one of embodiments 3 to 7, wherein the CORM is conjugated to a heavy chain of the antibody.

10. The conjugate of embodiment 9, wherein the CORM is conjugated to an Fc region of the antibody.

11. The conjugate of embodiment 9 or embodiment 10, wherein the CORM is conjugated to the C-terminus of the heavy chain.

12. The conjugate of any one of embodiments 1 to 11, wherein the CORM is site-specifically conjugated to the targeting moiety.

13. The conjugate of embodiment 12, wherein the targeting moiety comprises a non-natural amino acid to which the CORM is site-specifically conjugated.

14. The conjugate of any one of embodiments 1 to 13, wherein the CORM is covalently conjugated to the targeting moiety.

15. The conjugate of embodiment 14, wherein the CORM is conjugated to the targeting moiety via a linker.

16. The conjugate of embodiment 15, wherein the CORM is conjugated to the targeting moiety via a non-cleavable linker.

17. The conjugate of embodiment 16, wherein the non-cleavable linker is a thioether linker.

18. The conjugate of embodiment 17, wherein the thioether linker is a succinimidyl 4-(N-maleimidomethyl)cyclohexane-1-carboxylate (SMCC) linker.

19. The conjugate of embodiment 15, wherein the CORM is conjugated to the targeting moiety via a cleavable linker.

20. The conjugate of embodiment 19, wherein the cleavable linker is a peptide linker.

21. The conjugate of embodiment 20, wherein the peptide linker is a valine-citrulline (VC) linker.

22. The conjugate of any one of embodiments 15 to 21, wherein the linker comprises polyethylene glycol (PEG).

23. The conjugate of any one of embodiments 1 to 13, wherein the CORM is non-covalently conjugated to the targeting moiety.

24. The conjugate of embodiment 23, wherein the CORM is conjugated to the targeting moiety via a streptavidin-biotin interaction.

25. The conjugate of embodiment 24, wherein the CORM is biotinylated and the targeting moiety is conjugated to streptavidin.

26. The conjugate of any one of embodiments 1 to 25, wherein the CORM is a photoactivatable CORM (photoCORM).

27. The conjugate of embodiment 26, wherein the photoCORM comprises [Mn(CO)₃(phen)(4-pyAl)](CF₃SO₃), wherein phen is 1,10-phenanthroline, and 4-pyAl is pyridine-4-carboxaldehyde.

28. The conjugate of any one of embodiments 1 to 27, wherein the target cell is selected from the group consisting of: a cancer cell, an immune cell, and an endothelial cell.

29. The conjugate of embodiment 28, wherein the target cell is a cancer cell.

30. The conjugate of embodiment 29, wherein the cell surface molecule is a tumor antigen on the surface of the cancer cell.

31. The conjugate of embodiment 30, wherein the tumor antigen is selected from the group consisting of: 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), and trophoblast cell-surface antigen (TROP-2).

32. The conjugate of any one of embodiments 1 to 31, comprising two or more CORMs conjugated to the targeting moiety.

33. A pharmaceutical composition, comprising:
a conjugate of any one of embodiments 1 to 32; and
a pharmaceutically acceptable carrier.

34. The composition of embodiment 33, wherein the composition is formulated for parenteral administration.

35. A method comprising administering to an individual in need thereof an effective amount of a pharmaceutical composition of embodiment 33 or embodiment 34.

36. A method of treating cancer comprising administering to an individual having cancer an effective amount of a pharmaceutical composition of embodiment 33 or embodiment 34, wherein the targeting moiety specifically binds a tumor antigen present on cells of the cancer.

37. A kit comprising:
the conjugate of any one of embodiments 1 to 32; and
instructions for contacting cells with the conjugate to induce death of the cells.

38. A kit comprising:
the pharmaceutical composition of embodiment 33 or embodiment 34; and
instructions for administering an effective amount of the pharmaceutical composition to an individual in need thereof.

39. The kit of embodiment 38, wherein the kit comprises the pharmaceutical composition present in one or more unit dosages.

40. The kit of embodiment 38, wherein the kit comprises the pharmaceutical composition present in two or more unit dosages.

41. The kit of any one of embodiments 38 to 40, wherein the individual has cancer, the targeting moiety specifically binds a tumor antigen present on cells of the cancer, and the instructions are for administering to the individual a therapeutically effective amount of the pharmaceutical composition to treat the cancer.

42. A method, comprising:
conjugating a carbon monoxide (CO)-releasing molecule (CORM) to a targeting moiety that binds to a cell surface molecule on the surface of a target cell.

43. The method according to embodiment 42, wherein the conjugating comprises covalently conjugating the CORM to the targeting moiety.

44. The method according to embodiment 43, wherein the CORM is conjugated to the targeting moiety via a linker.

45. The method according to embodiment 42, wherein the conjugating comprises non-covalently conjugating the CORM to the targeting moiety.

46. The method according to any one of embodiments 42 to 45, wherein the conjugating comprises site-specifically conjugating the CORM to the targeting moiety.

47. The method according to embodiment 46, wherein the conjugating comprises site-specifically conjugating the CORM to a non-natural amino acid of the targeting moiety.

48. The method according to any one of embodiments 42 to 47, wherein the targeting moiety is an antibody.

49. The method according to any one of embodiments 42 to 48, wherein the targeting moiety specifically binds a tumor antigen.

50. The method according to any one of embodiments 42 to 49, wherein the CORM is a photoactivatable CORM (photoCORM).

The following examples are offered by way of illustration and not by way of limitation.

### EXPERIMENTAL

### Example 1—Eradication of Cancer Cells by Antigen-specific Delivery of Carbon Monoxide with a Family of Photoactivatable Antibody-photoCORM Conjugates

Reported herein is the successful conjugation of a photoCORM to monoclonal immunoglobulin G (IgG) antibodies to produce antibody-photoCORM conjugates (Ab-photoCORMs), and controlled delivery of CO to cancer cell cultures with high specificity. Utilizing different monoclonal antibodies, a family of Ab-photoCORMs was synthesized with the goal of localizing and delivering cytotoxic levels of CO to cancer cells expressing different tumor-specific surface antigens.

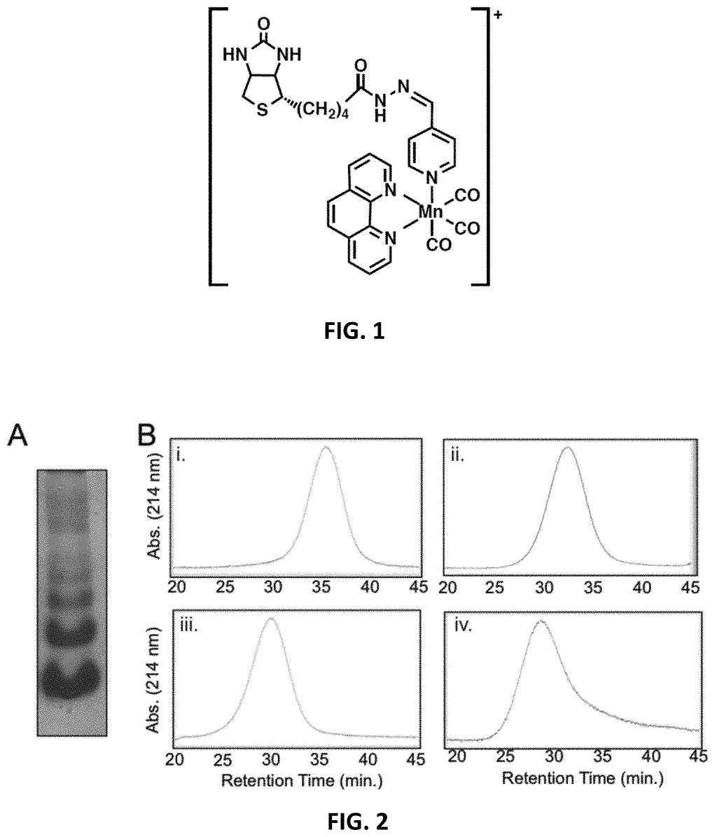

The present work utilized the photoCORM [Mn(CO)₃(phen)(4-pyAl)](CF₃SO₃) (where phen=1,10-phenanthroline, 4-pyAl=pyridine-4-carboxaldehyde) as the photoactivatable CO donor. Biotinylation of this photoCORM (FIG. 1, Complex 1) was achieved through reaction with biotin-hydrazide in trifluoroethanol at room temperature. The composition of Complex 1 was confirmed by electrospray ionization Fourier Transform mass spectrometry (ESI FTMS); (M⁺) m/z=666.13539 (calculated for C₃₁H₂₉N₇O₅SMn: 666.13313, Δppm=3.4 ppm, Δ mDa=2.2 (Figure S1), and ¹H NMR spectrum (Supporting Information). The Infrared spectrum of Complex 1 showed the presence of two ν_{C═O} bands at 2039 and 1939 cm⁻¹, characteristic of the manganese tricarbonyl moiety, and one μ_{C═O} band at 1685 cm⁻¹ derived from the biotin unit. Complex 1 was stable in phosphate-buffered saline in the dark for ˜48 h, releasing CO only upon illumination with low power (10 mW/cm²) visible light. Photorelease of CO was confirmed by myoglobin assay.

Previous studies from this laboratory have demonstrated that sufficient levels of CO, delivered from photoCORMs, can induce apoptotic cell death in a wide variety of cancer cells. Likewise, Complex 1 upon illumination with visible light, significantly reduced cell viability in two ovarian cancer cell lines OVCAR-5 and SKOV-3 (ED₅₀=48 and 25 μM respectively) assayed 24 h post-treatment.

A streptavidin-biotin strategy was used to link Complex 1 to IgG, exploiting the strong affinity (K_{d}=10⁻¹⁴M) and stability of the streptavidin-biotin interaction. The streptavidin-IgG conjugate was synthesized using a commercially available kit. Native gel electrophoresis (FIG. 2, panel A) and size exclusion chromatography revealed conjugation of a variable number of streptavidin molecules to IgG which was expected as per manufacturer's notes. Fractionation of crude streptavidin-IgG conjugates following size exclusion chromatography was performed to resolve and isolate antibodies conjugated with 1-4 streptavidin molecules (FIG. 2, panel B). These fractions were then pooled together (abbreviated hereafter as Complex 2) for cellular studies.

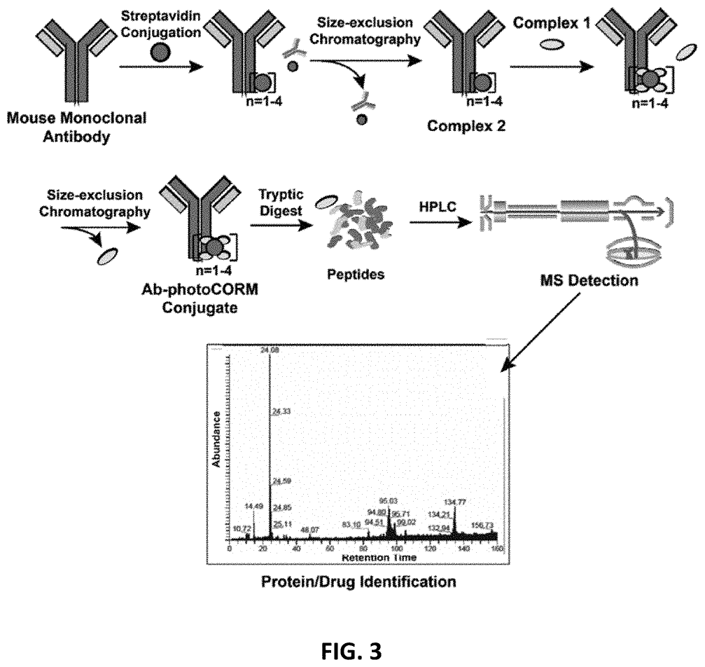

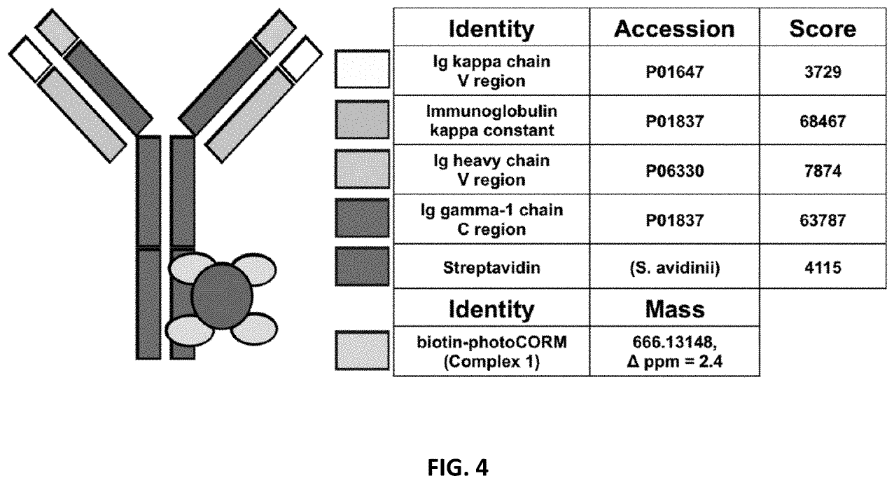

Reaction of Complex 2 with excess Complex 1 afforded the antibody-photoCORM conjugate (Ab-photoCORM) through a streptavidin-biotin interaction (FIG. 3). The Ab-photoCORM was then purified to remove any trace of unbound streptavidin, unconjugated IgG and unincorporated Complex 1 by size-exclusion chromatography (FIG. 3). Bottom-up proteomic analysis of the Ab-pohotoCORM confirmed the presence of streptavidin in the Ab-photoCORM (FIG. 4). Additionally, Complex 1 (M⁺) incorporated into the Ab-photoCORM was observed in the full MS scan (FIG. 4).

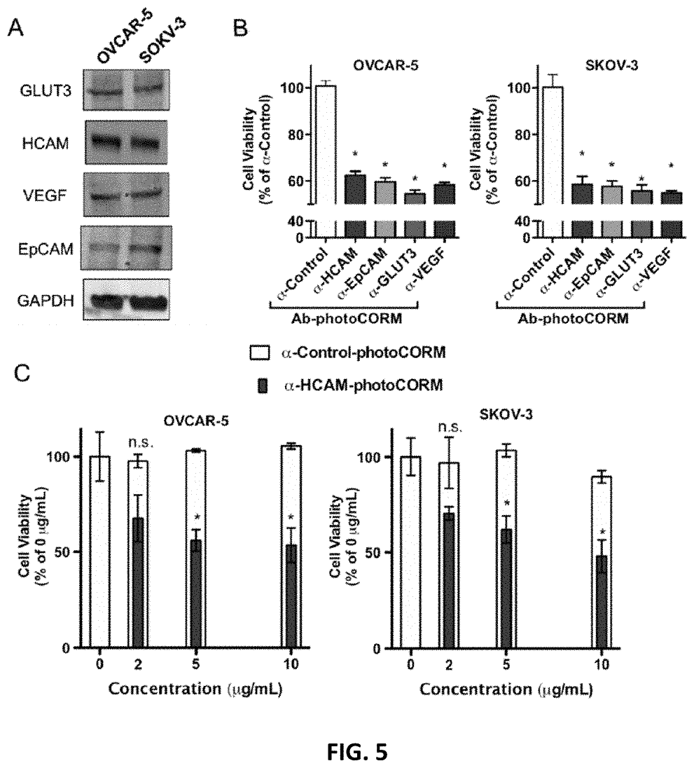

A family of Ab-photoCORM conjugates was synthesized (Table 1) using this synthetic strategy with commercially available mouse monoclonal IgG raised against four surface-expressed antigens implicated in ovarian cancer, namely homing cell adhesion molecule (HCAM), epithelial cell adhesion molecule (EpCAM), glucose transporter 3 (GLUT3), and vascular endothelial growth factor A (VEGF). Immunoblot analysis of whole cell lysates of cell line models utilized, OVCAR-5 and SKOV-3, confirmed the presence of the antigens recognized by the family of Ab-photoCORMs (FIG. 5, panel A). An Ab-photoCORM utilizing IgG not raised against any specific antigen (a-Control-photoCORM) was also synthesized for application in cell viability experiments in order to account for any non-CO-mediated effects of the antigen-specific Ab-photoCORMs.

TABLE 1
Family of antibody-photoCORM conjugates (Ab-photoCORMs) synthesized
from commercial antibodies, recognizing indicated human
cell surface antigens implicated in ovarian cancer.

| | | | Antibody- |
|---|---|---|---|
| | | | photoCORM |
| Original | | Streptavidin-IgG | conjugate |
| Mouse IgG | Epitote Recognized | (Complex 2) | (Ab-photoCORM) |
| HCAM | Homing cell adhesion | Complex 2-(α-HCAM) | α-HCAM- |
| (sc-7297) | molecule (human) | | photoCORM |
| EpCAM | Epithelial cell | Complex 2-(α-EpCAM) | α-HCAM- |
| (sc-53277) | adhesion molecule | | photoCORM |
| | (human) | | |
| GLUT3 | Glucose transporter 3 | Complex 2-(α-GLUT3) | α-GLUT3- |
| (sc-74399) | (human) | | photoCORM |
| VEGF-A | Vascular enothelial | Complex 2-(α-VEGF) | α-VEGF- |
| (365578) | growth factor A | | photoCORM |
| | (human) | | |
| Normal | None | Complex 2-(α-Control) | α-Control- |
| mouse IgG | | | photoCORM |
| (sc-2025) | | | |

The antigen-recognizing family of Ab-photoCORMs was assessed for their ability to localize and deliver cytotoxic levels of CO to OVCAR-5 and SKOV-3 cell cultures using a live-cell, immunosorbent assay. Adherent cells were first treated with 2 pg/mL of Ab-photoCORMs for 60 min in the dark and then washed 3 times with 1× PBS to remove any non-specific association. Next, fresh media was added to the cells and the cells were exposed to low-power visible light for 30 min for CO photorelease. After an incubation period of 24 h, cell viability was assessed by cellular reduction of MTT. The viability study clearly demonstrated that treatment of OVCAR-5 and SKOV-3 cells with Ab-photoCORM conjugates recognizing epitopes expressed in those ovarian cancer cell lines delivered cytotoxic levels of CO and dramatically decreased cell viability (FIG. 5, panel B). α-Control-photoCORM did not significantly reduce cell viability (FIG. 5, panel B), demonstrating that (a) CO alone was responsible for the cytotoxicity of the Ab-photoCORM complexes against the cancer cells, and (b) the presence of the correct antigen on the cancer cell surface was required for the targeted delivery of CO. Additionally, no significant cell death was observed either with light-inactivated Complex 1 or Complex 1 in the dark. Complex 2 also did not exhibit significant toxicity to both ovarian cancer cells.

In order to establish a dose-dependence of Ab-photoCORM in CO-induced cell death, the α-HCAM-photoCORM was utilized in a similar assay. As shown in FIG. 5, panel C, α-HCAM-photoCORM elicited dose-dependent decreases in cell viability of the OVCAR-5 and SKOV-3 compared to α-Control-photoCORM. It is important to note that in previous experiments, similar photoCORMs with no conjugation with antibodies exhibited CO-induced cell death at much higher concentrations (10-50 μM range) compared to the present study where cell death is evident in presence of hundreds of picomoles of CO. Taken together, these findings demonstrate the superior ability of the antigen-specific Ab-photoCORMs to accumulate onto ovarian cancer cells via recognition of surface proteins and deliver cytotoxic levels of CO in a much more efficient manner.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A conjugate, comprising:
a targeting moiety that binds to a cell surface molecule of a target cell; and
a carbon monoxide (C0)-releasing molecule (CORM) conjugated to the targeting moiety.

2. The conjugate of claim 1, wherein the targeting moiety is selected from the group consisting of: a polypeptide, an antibody, a ligand, an aptamer, a nanoparticle, and a small molecule.

3. The conjugate of claim 2, wherein the targeting moiety is an antibody.

4. The conjugate of claim 3, wherein the antibody is a monoclonal antibody.

5. The conjugate of claim 3, wherein the antibody is selected from the group consisting of: an IgG, Fv, single chain antibody, scFv, Fab, F(ab′)₂, or Fab′.

6. The conjugate of claim 3, wherein the CORM is conjugated to a heavy chain of the antibody.

7. The conjugate of claim 6, wherein the CORM is conjugated to an Fc region of the antibody.

8. The conjugate of claim 1, wherein the CORM is covalently conjugated to the targeting moiety.

9. The conjugate of claim 8, wherein the CORM is conjugated to the targeting moiety via a linker.

10. The conjugate of claim 1, wherein the CORM is non-covalently conjugated to the targeting moiety.

11. The conjugate of claim 10, wherein the CORM is conjugated to the targeting moiety via a streptavidin-biotin interaction.

12. The conjugate of claim 11, wherein the CORM is biotinylated and the targeting moiety is conjugated to streptavidin.

13. The conjugate of claim 1, wherein the CORM is a photoactivatable CORM (photoCORM).

14. The conjugate of claim 13, wherein the photoCORM comprises [Mn(CO)₃(phen)(4-pyAl)](CF₃SO₃), wherein phen is 1,10-phenanthroline, and 4-pyAl is pyridine-4-carboxaldehyde.

15. The conjugate of claim 1, wherein the target cell is selected from the group consisting of: a cancer cell, an immune cell, and an endothelial cell.

16. The conjugate of claim 1, wherein the target cell is a cancer cell and the cell surface molecule is a tumor antigen on the surface of the cancer cell.

17. The conjugate of claim 16, wherein the tumor antigen is selected from the group consisting of: 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), and trophoblast cell-surface antigen (TROP-2).

18. A pharmaceutical composition, comprising:
the conjugate of claim 1; and
a pharmaceutically acceptable carrier.

19. A method of treating cancer comprising administering to an individual having cancer an effective amount of a pharmaceutical composition of claim 18, wherein the targeting moiety specifically binds a tumor antigen present on cells of the cancer.

20. A method, comprising:
conjugating a carbon monoxide (CO)-releasing molecule (CORM) to a targeting moiety that binds to a cell surface molecule on the surface of a target cell.
